(19) United States
(12) Patent Application Publication
(10) Pub. No.: US 2003/0049671 A1
(43) Pub. Date: Mar. 13, 2003

(54) Surface-modified supporting materials for binding biological materials, methods for the production and use thereof

(76) Inventors: Timo Hillebrand, Berlin (DE); Peter Bendzko, Berlin (DE); Heike Matuschewski, Berlin (DE); Uwe Schedler, Berlin (DE); Thomas Thiele, Berlin (DE)

Correspondence Address: GABRIEL P. KATONA, GOODWIN PROCTER L.L.P., 599 LEXINGTON AVENUE, 40TH FLOOR, NEW YORK, NY, 10022, US

(21) Appl. No.: 10/254,107

(22) Filed: Jun. 3, 2002

Related U.S. Application Data
(63) Continuation of application No. PCT/DE00/04287, filed on Dec. 1, 2000.

(30) Foreign Application Priority Data
Dec. 3, 1999 (DE) 199 58 042.1

(51) Int. Cl. C12Q 1/68; G01N 33/53; G01N 33/542; C12M 1/34

(52) U.S. Cl. 435/6; 435/7.9; 435/287.2

(57) ABSTRACT

The invention relates to surface-modified supporting materials for binding biological materials, wherein the surface of said materials carries negatively charged functional groups.

This is a continuation of International Application No. PCT/DE00/04287 filed on Dec. 1, 2000.

The invention relates to surface-modified supporting materials for binding biological materials, methods for their production and use thereof for the isolation and purification of nucleic acids, especially of plasmid DNA. The inventive supports are characterized by a polymer layer on the surface, which has negatively charged functionalized groups.

The isolation and purification of biological materials, especially of polynucleotides play an important role in all areas of modem biochemistry and biotechnology. The demand for efficient methods for isolating and purifying nucleic acids is increasing constantly. Special attention is devoted to the isolation of plasmid DNA from bacterial lysates.

According to the state of the art, plasmid DNA from bacterial lysates can be isolated chemically with very simple methods. For this purpose, the pellet of bacteria is resuspended in a buffer consisting of glucose, tris-HCl and EDTA, subsequently lysed with an SDS/NaOH buffer and neutralized with a buffer consisting of potassium and sodium acetate. The neutralization reaction leads to complexing and subsequent precipitation of chromosomal DNA and proteins, which are pelletized by a centrifugation step. The resulting supernatant contains the plasmid DNA, which is precipitated by the addition of an alcohol, subsequently washed and dried and finally the plasmid DNA pellet obtained is hydrated in a tris buffer.

This method is very simple and relatively inexpensive and does not require any materials, which are ecologically and toxicologically harmful. However, it is difficult to automate this method, which furthermore is very time-consuming because of the many manual steps.

Commercially available methods for isolating plasmid DNA (also the fully automatic isolation of these materials) make use of very efficient membrane technologies for binding nucleic acids. Chaotropic ions are essential for binding nucleic acids. Those, skilled in the art, know that these chaotropic salts, as components of buffers, destroy the three-dimensional structure of hydrogen bonds. This leads to the weakening of intramolecular binding forces, which participate in the formation of spatial structures, such as secondary, tertiary or quaternary structures, in biological molecules. Due to these disorders of higher order structures of the aqueous milieu, it becomes possible for the nucleic acids to adsorb at the surface of mineral materials, especially of glass or silica particles. The solid phases, used according to the state of the art, exclusively are silica supports, diatomaceous earths or glasses, these materials being in the form of filter membranes or suspensions for the process of extracting nucleic acids. For isolating nucleic acids, they are always combined with solutions, which contain chaotropic salts, in reaction batches, in order to isolate or purify the desired nucleic acids. In the course of the reaction, chaotropic salts are added after the neutralization reaction as additional buffer components or are already a component of the neutralization buffer. The centrifuged, clear supernatant is then not subjected to an ethanol precipitation. Instead, it is brought together with the solid phase (glass materials, silica materials), to which it is bound, then washed and finally eluted once again form of the solid phase with a buffer of the lower ionic strength.

Admittedly, these methods can be carried out easily, save time and, if binding membranes are used, can be automated completely. However, because chaotropic buffer components are used, they are expensive and harmful to health and furthermore contaminate the environment.

The known chaotropic salts are also used as binding reaction component for the method described in DE 197 46 874 Al for isolating RNA, for which nucleic acids are bound using hydrophobic membranes.

Alternative methods for purifying DNA molecules using solid phase extraction, which does not employ chaotropic ions to bind the DNA, are also known. For these, the nucleic acids are bound to chemically modified solid phases, which are doped with positive ionic charges by chemical modification reactions. Accordingly, a bond is formed by Coulombic interactions between the positively charged surface of the membranes used and the negative ionic charge of the phosphate backbone of nucleic acids (U.S. Pat. No. 5,523,392 A; Purification of DNA on Aluminum Silicates and Phosphosilicates; U.S. Pat. No. 5,503,816 A; Silicate Compounds for DNA Purification; U.S. Pat. No. 5,674,997 A; DNA Purification on Modified Silicates; U.S. Pat. No. 5,438,127 A; DNA Purification by Solid Phase Extraction Using a PCl₃-Modified Glass Fiber Membrane; U.S. Pat. No. 5,606,046 A; DNA Purification by Solid-Phase Extraction Using Trifluorometric Acid-Washed Glass Fibers; U.S. Pat. No. 5,610,291 A: Glass fiber membranes modified by treatment with SiCl₄, AlCl₃ or BCl₃ and washing with NaOH to set as a DNA adsorbent; U.S. Pat. No. 5,616,701 A; DNA Purification by Solid-Phase Extraction Using a Hydroxide-Washed Fiberglass Membrane; U.S. Pat. No. 5,650,506 A; Modified Fiberglass Membranes Useful for DNA Purification by Solid Phase Extraction).

The principle of binding nucleic acids to positively charged solid phases, which is adequately known to those skilled in the art, is used. For many years already, it represents the standard application, for example, for DNA/RNA blotting techniques on positively charged nylon fibers.

However, the use of these membranes presupposes that the nucleic acids have already been isolated. Previously isolated nucleic acids can be bound by the positive, functional surfaces produced by Coulombic interactions at the membranes.

It is therefore an object of the invention to find and make available supporting materials, which make it possible to combine the classical chemistry for isolating biological materials, especially for isolating and purifying nucleic acids, with binding to a solid phase, without using dangerous material groups, such as chaotropic salts, and, moreover, offer the possibility of a highly efficient, automated method.

Surprisingly, and in contrast to previously suggested models, this object was accomplished by a fictionalization of the surfaces of supporting materials, wherein negative charges are present on the surface of the support. In combination with classical chemistry, it was surprisingly possible to realize binding of biological materials to a solid phase by these means. In particular, it was possible to utilize the negatively functionalized supports for isolating and purifying nucleic acids and especially for isolating and purifying plasmid DNA.

The invention is realized in accordance with the claims. Pursuant to the invention, a solid support is pretreated and brought into contact with monomer solutions of polymerizable acids or their derivatives, which are grafted to the support surfaces.

The support is pretreated preferably under alkaline conditions with a solution of a strongly basic material, preferably alkali hydroxides or amides, and an alcohol, the base and the alcohol being present in a ratio of 1:2 to 1:10,000 (0.01%). This process requires up to 24 hours and preferably about one to two hours. Subsequently the support is washed until the washings are neutral. After that, it is dried.

For the polymerization, the support is immersed in a monomer solution of the polymerizable acids in the presence of an initiator, exposed in the moist state to light, washed after the exposure and dried.

Accordingly, surface-modified supporting materials for binding biological materials, which are characterized by a layer, having negatively charged functional groups, on the surface of the support, are an object of the invention. Furthermore, a method for their production and their use for the isolation of nucleic acids, preferably of plasmid DNA, is a further object of the invention.

Within the sense of the invention, supporting materials are mineral/inorganic supporting materials, which may or may not be porous. Preferably, they are silica supports, diatomaceous earths or glass materials in the form of membranes, nonwoven fabrics, powders, granulates, spheres or particles, etc. However, as supporting materials, organic polymers, such as polypropylene, polyethylene, polyether sulfone, polystyrene, polyvinyl chloride, polyacrylonitrile, cellulose and its derivatives, polyamides, polyimides, polytetrafluoroethylene, polyvinylidene difluoride, polyvinylidene fluoride, polyester, polycarbonate, polyacrylates, polyacrylamide, etc., as well as copolymers or blends of polymers also come into consideration.

Pursuant to the invention, the layer with negatively charged groups on the surface of the support represents a polymer layer, which was formed with the polymerizerable acids or their mixtures. Preferably the polymer layer represents carboxylic acid, sulfonic acid and/or phosphoric acid derivatives, especially derivatives of acrylic acids, methacrylic acid, styrenesulfonic acid and styrenephosphoric acid and acrylamidopropanesulfonic acid or their mixtures.

In a special variation, the support is a nonwoven glass fabric, on which there is a polymer layer of the acrylic acid, a polymer layer of acrylate salts of the alkali metals or a polymer layer of 2-acrylamido-2-methylpropanesulfonate. In a different preferred variation, the support is in the form of a membrane of hollow glass fibers and, on which there is the polymer layer.

The inventive method is described in greater detail in the following. In the first step of the process, a convenient supporting material is immersed for a period of one minute to two hours, preferably for one hour, at room temperature in a solution of 0.1 g to 500 g of sodium hydroxide, potassium hydroxide or a different strongly basic material per 1000 g of an alcohol, preferably i-propanol. Subsequently, the supporting material is removed from the alcoholic solution and washed preferably with water until the washings are neutral, and dried, preferably for 30 minutes at 100° C.

In the subsequent, second step of the process, the pretreated support is coated with a photoinitiator, preferably benzophenone or its derivatives. As solvent for the initiator, ketones, alcohols, esters and ethers come into consideration. The concentration of the initiator preferably is 0.01 g/L to 0.5 g/L. The pretreated supporting material, charged with initiator, is dipped into a monomer solution, removed from the monomer solution and exposed to light in the moist state. As monomers, all polymerizable carboxylic acid, sulfonic acid and phosphoric acid derivatives are suitable, especially the derivatives of acrylic acid, methacrylic acid, styrenesulfonic acid and styrenephosphoric acid, acrylamidopropanesulfonic acid and also their mixtures. Alcohols, ketones, esters, ethers and especially water as well as mixtures of a these materials are used as solvent for the monomers. The concentration of the monomers preferably is 1 g/L up to 200 g/L, solutions with a monomer content of 50 g/L being particularly suitable.

As a source of light for the irradiation, preferably mercury vapor lamps, high-pressure and very high-pressure mercury vapor lamps, halogen lamps, tungsten lamps and lasers with emissions within the absorption range of the initiator are used. The exposure time depends on the intensity of the source of radiation and, depending on the power the latter, ranges from a fraction of a second to several hours.

After the exposure to light, the supporting material is washed. The solvents, which were used to prepare the monomer solution, are particularly suitable as washing liquid. The prepared supports subsequently are dried.

Surprisingly, due to the inventively coordinated conduct of the process corresponding to the two steps given, supporting materials are obtained, which are outstandingly suitable for isolating and purifying biological materials, especially plasmid DNA.

By means of the inventive process, the surface of the support is functionalized evidently because of the grafting polymerization with acid groups and their salts. However, these acid groups, especially because of the pretreatment of the surfaces of the support, are present as negatively charged acid ions. Unexpectedly, however, in spite of these negative groups, interactions with biological materials, especially interactions with plasmid DNA, are built up. In its variations, the inventively modified supporting material surprisingly makes a highly efficient isolation of polynucleotides possible. With that, the method of isolating nucleic acids can be automated completely, does not require any dangerous materials and is very cost effective in use. Alternatively, a cost-effective kit can be prepared preferably for the isolation of plasmid DNA from bacterial lysates.

Kits for plasmid DNA, which are used at the present time for methods, which can be automated, are based on the method for the preparative and analytical purification of DNA fragments from agarose gels, developed and described for the first time by Vogelstein and Gillespie (Proc. Natl. Acad. Sci. USA, 1979, 76, 615-619). The method combines the dissolving of the agarose, containing the DNA bands that are to be isolated, in a saturated solution of a chaotropic salt (sodium iodide) with binding the DNA to glass particles. The DNA, fixed to the glass particles, is subsequently washed with a solution of 20 mM tris HCl (pH 7.2), 200 mM sodium chloride, 2 mM EDTA and 50% v/v ethanol and finally dissolved from the support particles. Based on the well-known physicochemical principle of binding nucleic acids to silica or glass materials in the presence of chaotropic salts, a series of applications was described for the isolation of plasmid DNA and different supporting materials were used (such as glass milk, BIO 101, La Jolla, Calif., diatomaceous earth (Fa. Sigma) or also silica gels, DE 41 39 664 A1).

Aside from the solvents necessary for the extraction of plasmid DNA, the inventive kit contains an inventive supporting material, which can be inserted in the form of membranes in or on all conventional cartridges, test plates and wells.

Subsequently, the invention is explained in greater detail by means of examples without being limited to these.

### EXAMPLE 1

Preparation of a Fiberglass Nonwoven Material

The fiberglass nonwoven material in the DIN A4 format was immersed for a period of 1 hour at room temperature in a 100:1000 KOH/i-propanol solution. Subsequently, it was washed with water until the washings were neutral and dried for 30 minutes at 100° C. After that, the nonwoven material, so pretreated was coated with the benzophenone initiator (the concentration of the initiator was 0.15 moles/L). Acetone was used a solvent for the initiator.

The fiberglass nonwoven material, pretreated and charged with initiator, was then dipped in a monomer solution of acrylic acid in water. The concentration of the monomer was 50 g/L. The exposure to light was carried out with a Beltron UV dryer for a period of 20 minutes (corresponding to 20 cycles through the exposure sector).

After the exposure to light, the prepared nonwoven fiberglass material was extracted with methanol and water and subsequently dried.

### EXAMPLE 2

Analogously to Example 1, a fiberglass nonwoven material was immersed in a monomer solution of the potassium salt of methacrylic acid.

### EXAMPLE 3

Analogously to Example 1, a hollow fiber membrane of fiberglass was immersed in a monomer solution of 2-acrylamido-2-methylpropanesulfonate in water.

### EXAMPLE 4

Isolation of Plasmid DNA by Means of not Functionalized and Functionalized Nonwoven Materials

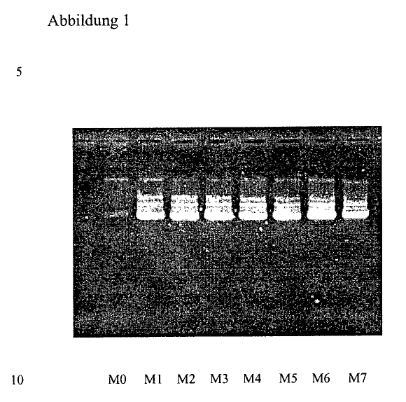

Plasmid DNA was isolated using the classical buffer, that is, without the previously essential chaotropic salts and by means of a not functionalized as well as various modified nonwoven fiberglass materials. The results are given in FIG. 1.

| | |
|---|---|
| MO | normal nonwoven material |
| M1 | modification of Example 1, |
| | reaction time: pretreatment: 10 minutes: 10 cycle exposure to light |
| M2 | modification of Example 1, |
| | reaction time: pretreatment: 30 minutes: 10 cycle exposure to light |
| M3 | Modification of Example 1, |
| | reaction time: pretreatment: 1 hour: 10 cycle exposure to light |
| M4 | Modification of Example 1, |
| | reaction time: pretreatment: 2 hours: 10 cycle exposure to light |
| M5 | Modification of Example 1, |
| | reaction time: pretreatment: 1 hour: 5 cycle exposure to light |
| M6 | Modification of Example 1, |
| | reaction time: pretreatment: 1 hour: 10 cycle exposure to light |
| M7 | Modification of Example 1, |
| | reaction time: pretreatment: 1 hour: 20 cycle exposure to light |

In each case, 2 mL of a bacterial suspension were transferred to a 2 mL reaction vessel and the cells were pelletized by centrifuging at 14,000 rpm for 1 minute. The pellet was resuspended in 100 μL of solution (25 mM of tris HCl; 10 mM of EDTA; 0.5 mg/mL of (Rnase A). Subsequently 300 μL of solution II (1% SDS/0.2 N NaOH) and 300 μL of solution III (3M potassium acetate; pH 5.2) were added. The solutions were mixed carefully and centrifuged for 8 minutes at 14,000 rpm. The clear, supernatant was transferred completely to the respective filter cartridge with the nonwoven material and centrifuged for 1 minute at 12,000 rpm. After the filtrate was discarded, the nonwoven material in the filter cartridge was washed twice by centrifuging with a washing buffer (70% ethanol, 100 mM NaCl; 15 mM tris HCl, 2 mM EDTA. The nonwoven material was then dried by centrifuging for 2 minutes. The plasmid DNA was eluted by the addition of 100 μL of an elution buffer (10 mM tris HCl), followed by centrifuging for 1 minute at 12,000 rpm.

Subsequently, 10 μL of the isolated DNA were analyzed on a 1% TAE agarose gel (FIG. 1).

As can be seen, only a very inadequate isolation of the plasmid can be carried out with the unmodified nonwoven fabric. On the other hand, with the modified nonwoven fabric M1 to M7, it is possible to realize very high yields and a qualitatively high-grade plasmid isolation. The effect is therefore clearly achieved by the inventive functionalization.

Key for FIG. 1

Gel electrophoretic representation of the isolated pDNA (in each slot, 1/20 of the eluate was applied; staining with ethidium bromide)

M0: unmodified nonwoven fabric

M1 to M7: modified nonwoven fabric

1. Surface-modified supporting materials for binding biological materials, wherein a layer on the surface carries negatively charged functional groups.

2. The supporting materials of claim 1 wherein the supporting material is a mineral, inorganic material.

3. The supporting materials of claim 2, wherein the supporting material is a mineral material, which is or is not porous.

4. The supporting materials of claim 3, wherein the supporting materials are silica supports, diatomaceous earth or glass materials, which preferably are present in the form of membranes, nonwoven materials, powders, granulates, spheres or particles.

5. The supporting materials of claim 1, wherein the supports are organic polymers, such as polypropylene, polyethylene, polysulfone, polyether sulfone, polystryene, polyvinyl chloride, polyacrylonitrile, cellulose and their derivatives, polyamides, polyimides, polytetrafluoroethylene, polyvinylidene difluoride, polyvinylidene fluoride, polyester, polycarbonate, polyacrylates, polyacrylamide as well as copolymers or blends of the polymers.

6. The supporting materials of one of the claims from 1 to 5, wherein the layer on the surface is a polymer layer, which has negatively charged groups.

7. The supporting materials of claim 7, wherein the polymer layer is formed from polymerizable carboxylic acids, sulfonic acids and phosphoric acids and their derivatives, preferably from derivatives of acrylic acid, methacrylic acid, styrenesulfonic acid and styrenephosphoric acid, acrylamidosulfonic acid and their mixtures.

8. The supporting materials of one of the claims 1 to 7 wherein the support is a nonwoven glass fabric, on which there is a polymer layer of acrylic acid.

9. The supporting materials of claims 1 to 7, wherein the support is a nonwoven glass fabric, on which there is a polymer layer of methacrylate salts of alkali metals.

10. The supporting materials of one of the claims 1 to 7, wherein the support is a hollow fiber membrane on which there is a polymer layer of 2-acrylamido-2-propanesulfonate.

11. A method for the production of solid supporting materials with functionalized surfaces of one of the claims 1 to 10, wherein a solid support is pretreated and subsequently brought into contact with polymerizable acids, which are graft polymerized onto the surface of the support.

12. The method of claim 11, wherein the support is pretreated for up to 24 hours under alkaline conditions with a solution of a strongly basic material, preferably alkali hydroxides or amides, and an alcohol, preferably i-propanol and the support subsequently is washed until the washings are neutral, after which it is dried.

13. The method of claim 12, wherein the base and the alcohol are present in a ratio of 1:2 to 1:10,000.

14. The method of one of the claims 11 to 13, wherein the polymerization is carried out in the presence of a photoinitiator, the support being immersed in a monomer solution of the polymerizable acids, taken out, exposed to the light in the wet state and, after the exposure, washed and dried.

15. The method of claim 14, wherein the monomer solution is used in a concentration of 1 g/L to 200 g/L and preferably with a monomer content of 50 g/L.

16. The method of claim 14 or 15, wherein water, alcohols, esters, ethers, ketones, or their mixtures and preferably water, methanol and acetone are used as solvent for the monomer solution.

17. The method of one of the claims of 11 to 16 wherein, mineral materials, such as silica supports, diatomaceous earths or glasses, which preferably are present in the form of membranes, nonwovens, powders, granulates, spheres or particles, function as solid supports.

18. The method of one of the claims 1 to 16, wherein polymers such as polypropylene, polyethylene, polysulfone, polyethersulfone, polystyrene, polyvinyl chloride, polyacrylonitrile, cellulose and its derivatives, polyamides, polytetrafluoroethylene, polyvinylidene difluoride, polyvinylidene fluoride, polyester, polycarbonate, polyacrylates, polyacrylamide and copolymers or blends of the polymers, function as supports.

19. The method of one of the claims 11 to 15, wherein carboxylic acids, sulfonic acids, phosphoric acids and their derivatives, and preferably derivatives of acrylic acid, methacrylic acid, styrenesulforic acid and styrenephosphoric acid, acrylamidosulfonic acid or their mixtures are used as polymerizable acids.

20. Surface modified supporting materials for binding biological materials produced by one of the claims 11 to 19.

21. The use of surface modified supporting materials with polymer layers, which have negatively charged functional groups, for the isolation of nucleic acids and preferably of plasmid DNA.

22. A kit for isolating and purifying plasmid DNA, comprising a) a functionalized supporting material of one of the claims of 1 to 10 and b) solutions for isolating plasmid DNA.
